(19) United States
(12) Patent Application Publication
(10) Pub. No.: US 2003/0133950 A1
(43) Pub. Date: Jul. 17, 2003

(54) Selective activation of Th1 or Th2 lymphocyte regulated immune response

(75) Inventors: Jacob Gabriel Michael, Saratoga, FL (US); Hans-Ulrich Petereit, Darmstadt (DE); Klaus Lehmann, Rossdorf (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: ROEHM GmbH & Co., KG, Darmstadt (DE)

(21) Appl. No.: 10/210,068

(22) Filed: Aug. 2, 2002

Related U.S. Application Data
(63) Continuation of application No. 09/757,311, filed on Jan. 8, 2001.
(60) Provisional application No. 60/174,994, filed on Jan. 7, 2000.

(51) Int. Cl. A61K 39/12; A61K 39/35; A61K 39/36; A61K 39/02; A61K 31/715; A61K 9/20; A61K 48/00

(52) U.S. Cl. 424/204.1; 424/234.1; 424/275.1; 424/465; 514/2; 514/44; 514/54

(57) ABSTRACT

The invention discloses methods for inducing a desired T helper lymphocyte regulated immune response by delivering an immunogen to a preselected region of the gastrointestinal tract of a subject. The invention finds application in the immunological and biomedical fields.

### CROSS-REFERENCES TO RELATED APPLICATIONS

This non-provisional patent application claims the benefit of the previously filed U.S. provisional patent application No. 60/174,994, filed Jan. 7, 2000, the text of which is hereby incorporated by reference.

### COPYRIGHT NOTICE

A portion of the disclosure of this patent document contains material which is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### FIELD OF THE INVENTION

This invention relates generally to a method of inducing or modulating an immune response in a subject by administering and delivering an immunogen to a preselected region of the gastrointestinal tract of the subject.

### BACKGROUND OF THE INVENTION

The immune system is a complex network of cells, tissues and organs that directly and indirectly target and ultimately destroy foreign substances. Of the various cells involved in mounting an immune response, lymphocytes are one type of white blood cell that have a crucial role. One type of lymphocyte is the B lymphocyte (B cell) that targets and indirectly destroys foreign substances by mounting a humoral immune response to produce antibodies against specific antigens. The other type of lymphocyte is the T lymphocyte (T cell) that targets and directly kills foreign substances by mounting a cell-mediated immune response. There are three major subtypes of T cells designated as T helper cells, T suppressor cells, and T cytotoxic cells.

T helper cells are of two principal types: Th1 and Th2 cells. They play a critical role in the activation and regulation of an immune response. If an immune response is controlled by Th1 cells, it results in the production of IgG2a antibodies in mice or their equivalent in other animal species and humans. IgG2a antibodies bind serum complement proteins and are effective at neutralizing viral and bacterial pathogens. Cytokines produced by Th1 cells activation are IL-2, IFN.gamma., IL-12, IL-18. These cytokines are involved in macrophage activation, which is important in antimicrobial and antiviral defenses and in the development of cytotoxic lymphocytes. On the other hand, Th1 associated immune responses can be associated with the inappropriate inflammatory responses often associated with autoimmune disease. Such an instance is in the pathology associated with myasthenia gravis. Th1 cytokine production is inhibitory to the activation of Th2 cells.

In contrast, Th2 cells help B cells mount a humoral immune response and help maintain T cytotoxic cells by producing growth factors needed by the T cytotoxic cells. Th2 cells are involved in production of IgG1, IgE and IgA antibodies in mice or their equivalent in different animal species and humans. IgG1 antibodies are important in antimicrobial and antiviral humoral defenses; IgE antibodies play a role in allergic diseases and asthma, their beneficial effect is linked to helminth and parasitic infections; IgA antibodies protect mucosal surfaces from infections. Th2 directed antibody responses may also be detrimental when the antibodies produced are reactive with “self” proteins. Autoimmune pathology associated with inappropriate antibody production is demonstrated in the case of rheumatoid arthritis. Several cytokines are produced as a result of Th2 cell activation. They are IL-4, IL-5, IL-10, IL-13 and while they support production of above-mentioned classes and subclasses of antibodies, they are antagonistic towards the activation of Th1 cells. Production of cytokines can be studied both in vivo and in vitro, their concentration is in constant flux and are produced by different cell populations besides lymphocytes (macrophage, NK cells, dendritic cells).

Immunization via the mucosal route, including the oral route, has been hampered by difficulty to achieve immune responses when administering the antigens in a non-viable form in absence of an adjuvant. Not only that immune response does not occur but that oral administration of the antigen may result in the induction of unresponsiveness.

Thus, there is a need for methods to induce a desired type of immune response and to downregulate an inappropriate response, especially through mucosal routes. Such a need is fulfilled by the instant invention.

### SUMMARY OF THE INVENTION

The present invention provides methods for inducing a desired type or downregulating an inappropriate type of T helper lymphocyte regulated immune response by delivering an immunogen to a preselected region of the gastrointestinal tract of a subject. In one aspect, the present invention provides a method for modulating, i.e., inducing or downregulating, a type of T helper lymphocyte-regulated immune response in a subject by orally administering to a subject immunogens which are coated with an aqueous enteric coating. The composition of the aqueous enteric coating enables its dissolution at the pH present in a preselected region of the GI tract, and allows for the release of the immunogens to that region of the GI tract. In one embodiment, the enteric coating allows release of immunogen in the duodenum and selectively induces a predominantly Th2 helper cell regulated immune response. In a related embodiment, the enteric coating used allows delivery of the coated immunogen to the lower portion of the GI tract and induces primarily a Th1 helper cell regulated immune response.

In another aspect, the invention provides a method of inducing multiple types of immune responses in a subject by administering to a subject multiple immunogenic compositions each containing at least one immunogen. The immunogen in each immunogenic composition is delivered to a different preselected region of the gastrointestinal tract of the subject and induces a different type of immune response. Each immunogenic composition is coated with a different enteric coating which dissolves at a different pH and releases the coated immunogen. Dissolution and immunogen release occur at that region of the GI tract that has a pH which is at the dissolution point for the enteric coating.

The invention further provides methods for inducing immune responses in a subject with immunogens that are labile at any pH below 7.0 (i.e., including the slightly acidic conditions of pH 5.5 to 7.0). The subject is administered such an immunogen that is encapsulated with an enteric coating which releases the immunogen only at a region of the gastrointestinal tract in which the immunogen is stable (i.e., where the pH is 7.0 or above). In some methods, the enteric coating is Eudragit® FS30D. In some methods, the region of the gastrointestinal tract in which the immunogen is released is jejunum, ileum, colon, or rectum.

### BRIEF DESCRIPTION OF THE DRAWINGS

FIG. 1 shows dissolution of FS30D- or L30D coated OVA particles under different pH conditions.

FIGS. 2A and 2B show dissolution of FS30D- or L30D-coated particles in the gastrointestinal tract.

FIG. 3 shows IgG, IgG1, and IgG2a levels in mice orally administered with L30D- or FS30D-coated particles.

FIGS. 4A and 4B show IgG, IgG1, IgG2a, and IgE levels of mice orally administered with L30D or FS30D coated OVA particles and subsequently challenged subcutaneously with OVA in IFA or CFA.

FIG. 5 shows T cell proliferation from mice orally administered with FS30D- or L30D-coated antigen and then challenged subcutaneously with OVA in IFA or CFA.

FIGS. 6A and 6B show cytokine levels in mice orally administered with L30D or FS30D coated particles and then challenged subcutaneously with OVA in CFA or IFA.

### DETAILED DESCRIPTION

Definitions

Unless defined otherwise, all technical and scientific terms used herein have the same meaning as commonly understood by those of ordinary skill in the art to which this invention pertains. The following references provide one of skill with a general definition of many of the terms used in this invention: Singleton et al., DICTIONARY OF MICROBIOLOGY AND MOLECULAR BIOLOGY (2d ed. 1994); THE CAMBRIDGE DICTIONARY OF SCIENCE AND TECHNOLOGY (Walker ed., 1988); and Hale & Marham, THE HARPER COLLINS DICTIONARY OF BIOLOGY (1991). As used herein, the following terms and phrases have the meanings ascribed to them unless specified otherwise. Although any methods and materials similar or equivalent to those described herein can be used in the practice or testing of the present invention, the preferred methods and materials are described. For purposes of the present invention, the following terms and phrases are intended to have the following general meanings as they are used herein:

The term “adjuvant”, as used herein, refers to any biological or chemical substance which, when administered with an immunogen, enhances the immune response against the immunogen, work, for example, by either concentrating antigen at a site where lymphocytes are exposed to the antigen or by inducing cytokines which regulate lymphocyte function. The adjuvant may be either a biological compound, a chemical compound that is therapeutically acceptable, or a combination of a biological and chemical compound. Examples of chemical adjuvants are water dispersible inorganic salts such as aluminum sulfate, aluminum hydroxide (alum) and aluminum phosphate. Examples of biological adjuvants are endogenous cytokines such as granulocyte-macrophage colony-stimulating factor (GM-CSF), tumor necrosis factor-.alpha. (TNF-.alpha.), interleukin-2 (IL-2), interleukin-4 (IL-4), interleukin-12 (IL-12) and interferon (IFN.gamma.), microorganisms such as BCG (bacille Calmette-Guerin), Corynebacterium parvum, and Bordetella pertussis, bacterial endotoxins such as cholera toxin B (CTB) or heat-labile toxin from E. coli (LT), lipopolysaccharide (LPS), and muramyldipeptide (N-acetylmuramyl-L-alanyl-D-isoglutamine (MDPI). Commercially available adjuvants such as DETOX.about.PC® are also available.

As used herein, the term “agent” includes any element, compound, or entity, including, but not limited to, e.g., pharmaceutical, therapeutic, pharmacologic, environmental or agricultural pollutant or compound, aquatic pollutant, cosmeceutical, drug, toxin, natural product, synthetic compound, or chemical compound.

The terms “antigen” or “immunogen” are broadly used herein to encompass any chemical or biological substance that elicits an immune response when administered to an animal. While an immunogen is frequently a protein, it may also be a nucleic acid, glycoprotein or polysaccharide. For the purpose of the present invention, immunogens include but are not limited to the following: an allergen, a killed bacterium or a bacterial component, a killed virus or a viral component, a peptide, a protein fragment, a protein, a glycoprotein, a gene, a gene fragment, a DNA, an RNA, a polysaccharide or lipopolysaccharide and any combinations of these substances. Examples of allergens include allergenic proteins and digested fragments thereof such as pollen allergens from ragweed, rye, June grass, orchard grass, sweet vernal grass, red top grass, timothy grass, yellow dock, wheat, corn, sagebrush, blue grass, California annual grass, pigweed, Bermuda grass, Russian thistle, mountain cedar, oak, box elder, sycamore, maple, elm and so on, dust, mites, bee and other insect venoms, food allergens, animal dander, microbial vaccines which in turn include viral, bacterial, protozoal, nematode and helminthic vaccines and their various components such as surface antigens, including vaccines which contain glycoproteins or proteins, protein fragments, genes or gene fragments prepared from, for example, Staphylococcus aureus, Streptococcus pyogenes, Streptococcus pneumoniae, Neisseria meningitidis, Neisseria gonorrhoeae, Salmonellae species, Shigellae species, Escherichia coil, Klebsiellae species, Proteus species, Vibrio cholerae, Helicobacter pylori, Pseudomonas aeruginosa, Haemophilus influenzae, Bordetella pertussis, Mycobacterium tuberculosis, Legionella pneumophila, Treponema pallidum, and Chlamydiae species, tetanus toxoid, diphtheria toxoid, influenza viruses, adenoviruses, paramyxoviruses, rubella viruses, polioviruses, hepatitis viruses, herpesviruses, rabies viruses, human immunodeficiency viruses, and papilloma viruses, in addition to protozoal parasites such as Toxoplasma gondii, Pneumocystis carinii, Giardia lamblia, Trichomonas vaginalis, Isospora beeli, Balantidium colt, Blastocystis hominis, and the various species of Entamoeba, Amebae, Plasmodium, Leishmania, Trypanosoma, Babesia, Cryptosporidium, Sarcocystis, and Cyclospora, as well as nematodes and helminths of the various species of trematodes, flukes, cestodes and visceral larvae.

Te term “autoimmune disease” refers to a spontaneous or induced malfunction of the immune system of mammals in which the immune system fails to distinguish between foreign immunogenic substances within the mammal and/or autologous (“self”) substances and, as a result, treats autologous (“self”) tissues and substances as if they were foreign and mounts an immune response against them. Autoimmune disease is characterized by production of either antibodies that react with self tissue, and/or the activation of immune effector T cells that are autoreactive to endogenous self antigens. The main immunopathologic mechanisms by which autoimmune diseases are mediated include: 1) autoantibodies are directed against functional cellular receptors or other cell surface molecules, and either stimulate or inhibit specialized cellular function with or without destruction of cells or tissues; 2) autoantigen—autoantibody immune complexes form in intercellular fluids or in the general circulation and ultimately mediate tissue damage; and 3) lymphocytes produce tissue lesions by release of cytokines or by attracting other destructive inflammatory cell types to the lesions. These inflammatory cells in turn lead to production of lipid mediators and cytokines with associated inflammatory disease.

As used herein, the term “enteric coating” means a coating surrounding the core, the solubility of the coating being dependent on the pH in such a manner that it prevents the release of the drug in the stomach but permits the release of the drug at some stage after the formulation has emptied from the stomach. The term “pH-sensitive enteric coating” respectively means a polymer the solubility of which is dependent on the pH. For example, a pH-sensitive enteric coating may be insoluble in gastric juice but dissolves at some stage after the formulation has emptied from the stomach. The term “pH dissolution point” means the pH value in which the pH-sensitive enteric polymer substantially begins to dissolve. The term delayed-release coating refers to a coating which dissolves as a result of mechanical abrasion or chemical interaction and releases antigen after a period of time. The term controlled-release coating refers to a coating which may be enteric or delayed release or which is dependent on some other variable (i.e. a particular enzyme or enzyme concentration) to determine the point of dissolution within the gastrointestinal tract.

As used herein, the phrases “FS30D coated immunogenic composition” or “FS30D coated antigen” (or equivalent phrases) refer to a immunogenic preparation which contains at least one immunogen encapsulated with an FS30D enteric coating. Prior to encapsulation with the enteric coating, the immunogen may also be microencapsulated on a pharmaceutically inert particle along with a stabilizing agent and a binding agent. As used herein, the phrases “FS30D coated antigen” or “FS30D coated immunogenic composition” are used interchangeably with “FS30D coated immunogenic particle” or “FS30D coated particle”. Similarly, the phrases “L30D coated immunogenic composition,” “L30D coated antigen,” “L30D coated immunogenic particle,” or “L30D coated particle” are used interchangeably and refer to the same immunogenic preparation except that the enteric coating is prepared with the L30D composition.

As used herein, the term “formulations” encompass both the different percentage compositions and different physicochemical compositions of the immunogenic compositions, such as size, coatings, polymers, plasticizers, anti-stick agents, anti-foam agents, antistatic agents, potentiating agent(s) and excipients.

As used herein, the term “inflammation” refers to both acute responses (i.e., responses in which the inflammatory processes are active) and chronic responses (i.e., responses marked by slow progression and formation of new connective tissue). Acute and chronic inflammation may be distinguished by the cell types involved. Acute inflammation often involves polymorphonuclear neutrophils; whereas chronic inflammation is normally characterized by a lymphohistiocytic and/or granulomatous response. Inflammation includes reactions of both the specific and non-specific defense systems. A specific defense system reaction is a specific immune system reaction response to an antigen (possibly including an autoantigen). A non-specific defense system reaction is an inflammatory response mediated by leukocytes incapable of immunological memory. Such cells include granulocytes, macrophages, neutrophils and eosinophils. Examples of specific types of inflammation are diffuse inflammation, focal inflammation, croupous inflammation, interstitial inflammation, obliterative inflammation, parenchymatous inflammation, reactive inflammation, specific inflammation, toxic inflammation and traumatic inflammation.

As used herein, the phrase “inducing an immune response” includes eliciting an immune response as well as modulating, selectively stimulating, and/or enhancing either a general or selective immune response.

As used herein, the terms “inert material” or “inert particle” refer to a pharmaceutically inert material substrate onto which the solution of one or more immunogens and an optional stabilizing agent may be applied to, for example by spraying. The mixture may then be coated with an aqueous enteric coating. The inert material may encompass a variety of shapes and forms such as a bead, a sphere, a powder, a crystal, or a granule. In one embodiment, a nonpareil, defined as a small round particle of a pharmaceutically inert material, may be used. One such nonpareil is available under the brand name Nupareils® (CHR Hansen Ingredient Technology, Vineland, N.J.). In other embodiments, a silica powder, sugar crystal or salt crystal may be used.

As used herein, the term “lower gastrointestinal tract” encompasses jejunum, ileum, colon, and rectum.

As used herein, the term “mucoadhesive agents” is defined as a substance which adheres to the mucosa of the gastrointestinal tract of an animal. A mucoadhesive agent such as Lycopersicon esculentum lectin (tomato lectin) or Chitosans-like N-trimethyl chitosan chloride binds to sugars and form glycoconjugates at site-specific areas of the intestines. A mucoadhesive agent can also function as a binding agent which binds an immunogen to an inert material. For example, 1-10% of polyvinylpyrrolidone can be used to bind a therapeutic protein to nonpareils and act as a mucoadhesive agent for the protein during the passage through the gastrointestinal tract.

The term “potentiating agent,” as used herein, refers to agents that enhance the antigenicity of other immunogens. A potentiating agent thus indirectly stimulates an immune response. Examples of potentiating agents include adjuvants, mucoadhesive agents, and promoting agents.

The term “physiological activity,” in reference to an organism is defined herein as any normal processes, functions, or activities of a living organism.

As used herein, the term “prophylactic activity” is an activity of, for example, an agent, gene, nucleic acid segment, pharmaceutical, substance, compound, or composition which, when administered to a subject who does not exhibit signs or symptoms of a disease or exhibits only early signs or symptoms of a disease, diminishes, decreases, or prevents the risk in the subject of developing pathology.

The phrase “promoting agents” is defined herein as formulation ingredient(s) that promote uptake, transport or presentation of antigen(s), adjuvants, or haptens thereby enhancing the desired immune response. Examples of promoting agents are glycoproteins, lipoproteins, bile salts, fatty acids, phospholipids, glycolipids, triglycerides, and cholesterol, cyclodextrins, glycerol, among others. All of the above potentiating agents may be incorporated into the immunogenic composition singly, in combination, or as part of covalent or noncovalent complexes.

As used herein, the term “stabilizing agent” refers generally to therapeutically inactive, water soluble agent that acts to protect the immunogen during a step in the formulation of the immunogen and/or during a subsequent coating step. Examples of stabilizing agents may include sugars (i.e. lactose, mannitol and trehalose) or cellulosic compounds (i.e. ethylcellulose or hydroxypropyl methylcellulose) or polyethylene glycol or a stabilizing protein (i.e. human serum albumin) or any of several compounds which are generally recognized as providing a stabilizing effect upon solid oral dosage forms and particularly upon proteins.

The term “subject” as used herein includes humans and animals, including mammals and non-mammals.

The term “therapeutic immunogen or therapeutic agent” is defined herein as one that alleviates a pathological condition or disease. Therapeutic agents that may be used in the present invention include, but are not limited to, immunogenic agents and gene therapy agents. A prophylactic agent is defined herein as one that either prevents or decreases the severity of a subsequently acquired disease or pathological process. An example of a prophylactic agent is a vaccine against a microbe causing an infectious disease.

The term “therapeutic activity” is defined herein as any activity of e.g., an agent, gene, nucleic acid segment, pharmaceutical, therapeutic, substance, compound, or composition, which diminishes or eliminates pathological signs or symptoms when administered to a subject exhibiting the pathology. The term “therapeutically useful” in reference to an agent means that the agent is useful in diminishing, decreasing, treating, or eliminating pathological signs or symptoms of a pathology or disease.

As used herein, the phrase “Th1 lymphocyte regulated immune response” (or equivalent phrases) refers to immune response that is predominantly regulated by Th1 helper cells. For example, a Th1 cell regulated immune response may lead to the production of predominantly IgG2a antibodies in mice or their equivalents in other animal species and humans. Cytokines produced in a Th1 cell regulated immune response may include any or all of the following: IL-2, IFN.gamma., IL-12, IL-18 cytokines. The phrase “Th2 lymphocyte regulated immune response” (or equivalent phrases) refers to an immune response that is predominantly regulated by Th2 helper cells. For example, a Th2 cell regulated immune response may result in the production of predominantly IgE, IgG1, and IgA antibodies or their equivalents in different animal species and humans. Cytokines produced in a Th2 cell regulated immune response may include IL-4, IL-5, IL-10, and IL-13.

As used herein, the term “trans-intestinal release coating” refers to an aqueous coating formulated to be insoluble under gastric conditions and in the upper portion of the small intestine but dissolves in the lower portion of the gastrointestinal tract. An example of such a coating is Eudragit® FS-30D (Rohm America Inc., Somerset, N.J.) which is an aqueous dispersion of a copolymer of methacrylic acid, methyl acrylate and methyl methacrylate. FS30D coating has a pH dissolution point of about 7.0, a pH which is typically found throughout the intestine in the region beyond the duodenum. The term “duodenal release coating” refers to an coating which is formulated to dissolve in the uppermost portion of the small intestine (duodenum). An example of such a coating is Eudragit® L-30 D-55 (Rohm America Inc., Somerset, N.J.) prepared from polymethacrylic acid and ethylacrylate [hereinafter referred to as “L30D”]. It has a pH dissolution point of about 5.5 and dissolves in the duodenum.

It was discovered during the course of the present invention that orally administered antigen will activate predominantly either T helper 1 lymphocytes or T helper 2 lymphocytes, depending on the region of the gastrointestinal tract in which the antigen-lymphoid tissue interaction occurs. When Eudragit L30D, a pH 5.5 sensitive enteric coating composition, was used to encapsulate an antigen to be orally administered, the antigen passes in intact form from the stomach to duodenum. The enteric coating protects the antigen from low pH and peptic digestion in the stomach. In duodenum where pH is substantially higher, the antigen is released and has the opportunity to interact with antigen presenting cells in the Peyer's patches and other components of the mucosal immune system. The immune response to encapsulated antigens with this pH 5.5 sensitive coating induces a Th2 regulated immune response as evidenced by production of IgG1, IgE, IgA and the cytokine IL-4.

On the other hand, the Eudragit® FS30D coating, which consists of a mixture of methacrylic and methylacrylic components, will dissolve and allow encapsulated antigen to be released only when the pH is above 7. Since the pH within the region of the intestine encompassing the jejunum to the rectum (but not the duodenum) is within this range, the release of the antigen would occur gradually. It is expected that some antigen release will occur even in the large intestine and that this release will enable intact antigen to interact with the lymphoid tissue (Peyer's patches) along the entire lower intestine. However, when an antigen encapsulated with Eudragie FS30D was orally administered to experimental animals (mice), the result was, surprisingly and unexpectedly, characteristic of a T helper 1 cell regulated immune response as indicated by the prevalent IgG2a production and the suppressed IgG1 and IgE production.

Accordingly, the present invention provides methods for inducing or downregulating a type of T helper lymphocyte regulated immune response by administering and delivering an antigen to a preselected region of the GI tract. In one aspect, the present invention provides methods for orally administering an immunogenic composition which allows the release of the immunogen at a preselected region of the GI tract. The chemical composition of the enteric coating may be formulated to dissolve, and thus release the immunogen, at a particular pH in the small intestine for an optimally selective T cell response. Alternatively, the enteric coating may be formulated to release the immunogen after encountering sufficient mechanical and/or chemical erosion or to release antigen when it encounters an enzyme or enzyme concentration that is unique to a particular region of the gastrointestinal tract.

In preferred embodiments, the immunogenic composition utilizes an aqueous enteric coating which will resist dissolution in the acidic medium of the stomach and will dissolve in the environment of the small intestine. The enteric coatings can have different compositions each having a different pH dissolution point. Using an enteric coating which will dissolve in a preselected region of the GI tract where the pH condition is comparable to the pH dissolution point of that coating, the coated immunogen will be released in that preselected region and selectively activate Th1 or Th2 cell regulated immune response.

Further, the present invention also finds applications in inducing immune responses with immunogens that are acid labile at any pH below 7.0. Such immunogens are acid labile in even slightly acidic conditions (between pH 5.5 and 7.0). For example, influenza viral antigens, are acid labile in that they are very pH sensitive and lose immunogenicity in any environment below pH 7.0. When coated with the Eudragit® L30D55 coating and orally administered to a subject (e.g., a human being), such immunogens are released in the duodenum and become degraded in the slightly acidic environment. As a result, these antigens cannot illicit intended immune responses in the subject. To protect immunogenicity of such antigens, they can be encapsulated with an enteric coating which releases the antigens only at pH 7.0 or above. One example of such coating is Eudragit® FS30D. When the antigens are released from such an coating at the lower portion of the intestine (e.g., from jejunum to the rectum) where the pH is higher than 7.0, the antigens remain stable and immunogenic.

Preparation of Immunogenic Composition

Unless specifically indicated otherwise, all percentages regarding immunogenic composition are given in terms of the weight of the ingredient relative to the total weight of the encapsulated immunogenic composition.

The present invention can be used to selectively induce Th1 or Th2 lymphocyte regulated immune response against various kinds of immunogens or antigens. Examples of immunogens that can be used for preparing immunogenic compositions according to the invention include, but are not limited to, an allergen, a killed bacterium or a bacterial component, a killed virus or a viral component, a peptide, a protein fragment, a protein or glycoprotein, a DNA, an RNA a polysaccharide or lipopolysaccharide, or any combinations of them.

To prepare the immunogenic composition of the present invention, the immunogen, bound to an inert core, may be directly coated with a coating of the desired composition. Alternately, the first step may be to form an aqueous solution of the immunogen with an optional stabilizing agent to provide physical protection for the immunogen and apply both to the inert core prior to enteric coating. Methods of preparing microencapsulated immunogenic compositions for oral administration are a well known in the art. See, e.g., U.S. Pat. Nos. 5,591,433, 5,609,871, 5,629,001, and 5,783,193. Similar methods are also described in WO 99/45904.

When a stabilizing agent is to used, it is added at a concentration of from about 0.1% to about 10%, with a concentration of about 5% being preferred. If the immunogen solution has a low viscosity, it may be desirable to add from about 1% to about 10% of polyvinyl pyrrolidone or other binding agents such as hydroxypropylcellulose or hydroxypropylmethylcellulose to bind the immunogen to the inert material.

The solution of one or more immunogens and an optional stabilizing agent is then applied, for example by spraying, to a pharmaceutically inert material substrate, hereinafter termed an inert material. The inert material may encompass a variety of shapes and forms such as a bead, a sphere, a powder, a crystal, or a granule. In one embodiment, a nonpareil pharmaceutically inert material may be used. One such nonpareil is available under the brand name Nupareils® (CHR Hansen Ingredient Technology, Vineland, N.J.). In other embodiments, a silica powder, sugar crystal or salt crystal may be used.

In one embodiment, the immunogenic compositions of the present invention comprise the antigen and an enteric coating, e.g., a trans-intestinal release coating or a duodenal-release coating. Many coating apparatuses can be used to coat the immunogen onto the inert material, including Glatt® brand fluid bed coaters and various other brands of Wurster type fluid bed coaters (NIRO, Vector, Fluid Air, etc.). Coating conditions and times vary depending on the apparatus and coating viscosity; however, coating must generally be conducted at temperatures less than about 50.degree. C., and preferably less than about 35.degree. C., to avoid denaturation of a protein immunogen.

To prepare the enteric coating for the immunogen or the dry immunogen-coated inert materials, one or more layers of acid stable polymers are used. The coating of one or more polymers may be applied in a similar manner and with similar equipment as the coating steps previously described.

To selectively induce T helper cell response, the enteric coating must render the immunogen resistant to degradation in the acid environment of the stomach. It should be able to dissolve and thus release the immunogen at a particular pH in the small intestine for an optimally selective T cell response. This could be achieved by specifically formulating the chemical composition of the enteric coating. In accordance with the present invention, the enteric coating is preferably a water-based emulsion polymer such as ethylacrylate methacrylic acid copolymer. Examples of the enteric coating composition include, but are not limited to Eudragit® L-30D and Eudragit FS-30D (Rohm America Inc., Somerset, N.J.). Eudragit L-30D has a molecular weight of about 250,000 and is generally supplied as a 30% w/v aqueous dispersion. It dissolves in an environment where the pH is above about 5.5. Eudragit® FS-30D is a 30% aqueous dispersion of a copolymer of methacrylic acid, methyl acrylate and methyl methacrylate and becomes soluble at a pH above 7. The enteric coatings produced with these compositions allow the microencapsulated immunogen to be orally administered without being released from the immunogenic composition until encountering a specific region of the gut where the pH condition allows the coating to dissolve.

The coating composition may also be combined with a plasticizer to improve the continuity of the coating. Several well known plasticizers may be used, with triethylcitrate (Morflex Inc., Greensboro, N.C.) preferred. Also, an antisticking agent, e.g., Talc (about 3.0%), may be added to prevent the particles from sticking to each other. In addition, an antifoaming agent (about 0.0025%) such as sorbitan sesquioleate (Nikko Chemicals Co. Ltd., Japan) or silicone can also be added. An antistatic agent (about 0.1%) such as Syloid 74FP (Davison Chemical Division, Cincinnati, Ohio) can be added. The inert materials containing the immunogen, the optional stabilizing agent or agents and other formulation ingredients are dried and may be coated with the enteric coating as previously described. The coating solution is about 30% to about 75% polymer, about 0% to about 10% plasticizer, about 0% to about 3% talc, about 0% to about 0.0025% antifoaming agent, about 0% to 3% antistatic agent and water. Although the enteric coating is essentially aqueous in nature, it may contain a minimal amount of organic solvents. However, it is generally preferable that there be no organic solvents in amounts which can fully denature the immunogen so that it no longer contains any of the antigenic determinants of the native molecule.

Potentiating Agents

In an alternative embodiment, a potentiating agent may be added to increase the immunogenicity of the protein. The potentiating agent may be added to the aqueous dispersion or solution of immunogen prior to coating onto the inert material. Alternatively, the potentiating agent may be added to non-immunogen bound inert materials. Generally, about 1% to about 10% of potentiating agent is added. The potentiating agent may be bound to the same inert material as the immunogen. Alternatively, the potentiating agent may be bound to a first inert material and the immunogen may be bound to a second inert material, such that the potentiating agent may be applied to non-immunogen bound inert materials.

Dosage and Mode of Administration

The immunogenic compositions of the present invention can be used to treat or alleviate symptoms associated with various diseases and disorders such as allergy, autoimmune diseases, and inflammation. Subjects with such diseases or disorders can be treated by using immunogenic compositions of the present invention to modulate an ongoing or potential immune response which is inappropriate to the subject, such as in the case of an autoimmune disease by inducing either a Th1 or Th2 regulated immune response.

The immunogenic compositions of the present invention can comprise immunogen encapsulated with an aqueous enteric coating, timed-release coating, or controlled-release coating. Also, the immunogen may be encapsulated first with a timed-release coating (or a controlled-release coating) followed by encapsulation with an enteric coating. Release of immunogen from such encapsulated immunogenic compositions can be made dependent on the composition of the timed-release coating (or controlled-release coating).

The immunogenic compositions of the present invention may conveniently be presented in unit dosage form and may be prepared by any methods well known in the art of pharmacy. See, e.g., Remington's Pharmaceutical Sciences (17th ed.), Mack Publishing Co., Easton, Pa.; Avis et al (eds.) (1993). The immunogenic compositions are administered in a dosing schedule to induce a desired T helper cell regulated immune response. The immunogenic compositions are preferably administered orally such as by gavage or feeding. Dosing may be consecutive or intermittent and at various times and in various formulations.

In one embodiment, an administered dose may contain a number of single immunogenic compositions each consisting of an enteric coating, timed-release coating, or controlled-release coating, at least one immunogen and, if added, the potentiating agent. Depending on the desired immune response to be induced, the various single immunogenic compositions of the administered dose may have the same coating or different coatings. They may also have the same formulation or different formulations of polymers, plasticizers, binding agents, anti-stick agents, anti-foam agents, antistatic agents, potentiating agent(s) and excipients, and/or the same inert material composition and size or different inert material compositions and sizes. Alternatively, the dose may be formulated to contain a combination of inert materials with one or more immunogens and, if added, the potentiating agent(s) in separate inert materials. If formulated with the immunogen and potentiating agent(s) in separate inert materials, the separate immunogenic compositions of the administered dose may have the same enteric coatings or different enteric coatings, the same formulations or different formulations of polymers, plasticizers, binding agents, anti-stick agents, anti-foam agents, antistatic agents, potentiating agent(s) and excipients, and/or the same inert material compositions and sizes or different inert material compositions and sizes. These various combinations and permutations of inert material size, inert material composition, coating, and formula composition help to achieve selective distribution and presentation of the antigen along the GI tract upon administration of the immunogenic compositions.

The immunogenic compositions may be placed in gel capsules for oral administration to humans or other mammals. Dosage will depend on the subject, the purpose, and the course of the treatment. For example, dosage for allergens may be different from the dosage used in immunotherapy by injection.

The following examples are provided to further illustrate the present invention. They are not included to limit the invention in any way.

### EXAMPLE 1

Dissolution of FS30D- or L30D Coated OVA Particles.

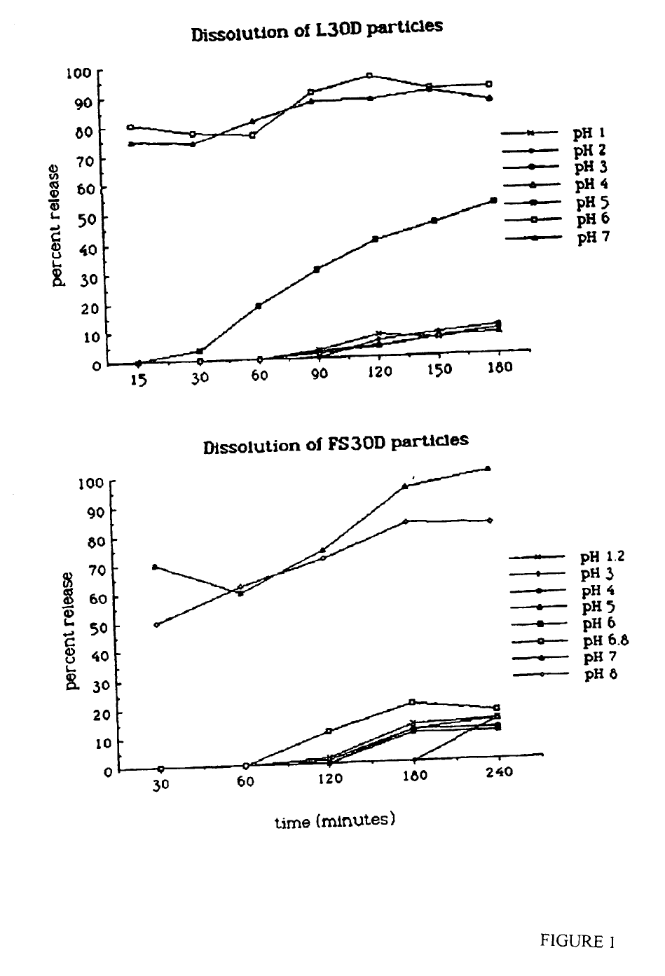

Hen egg albumin (OVA) was encapsulated with either L30D or FS30D composition. Dissolution of the immunogenic particles was measured over a course of 3 or 4 hours. The pH values used for L30D coated particles were from pH 1 to pH 7, while those for FS30D coated particles ranged from pH 1 to pH 8. Percentage of release was determined in a modified in-vitro dissolution assay. The results shown in FIG. 1 indicate that the FS30D coating do not dissolve when the pH is below 7 while the L30D coated particles begin breaking down at pH 5.

### EXAMPLE 2

Dissolution of FS30D- or L30D-Coated Particles in the Gastrointestinal Tract

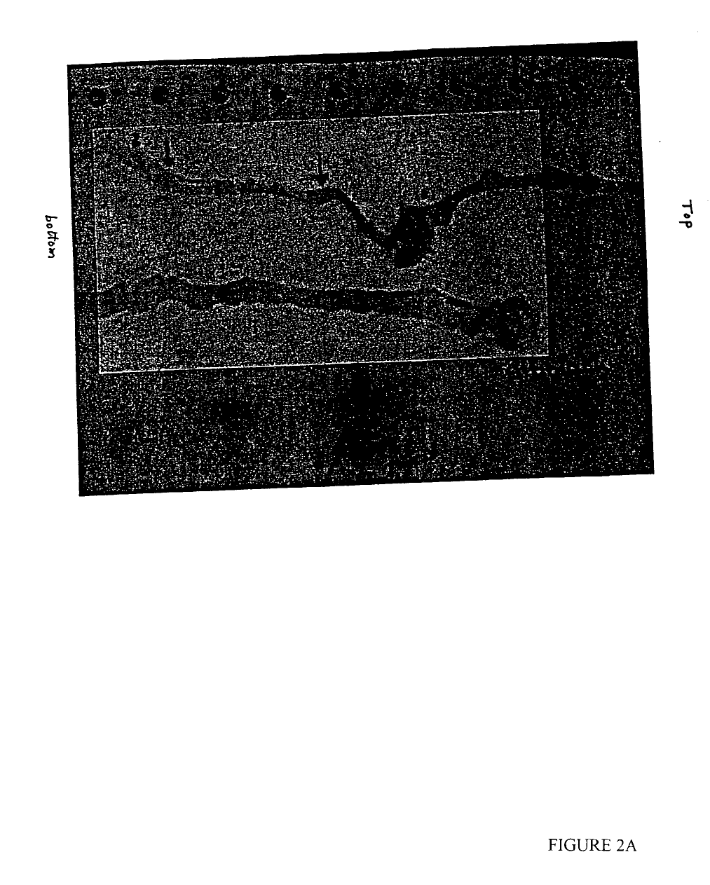

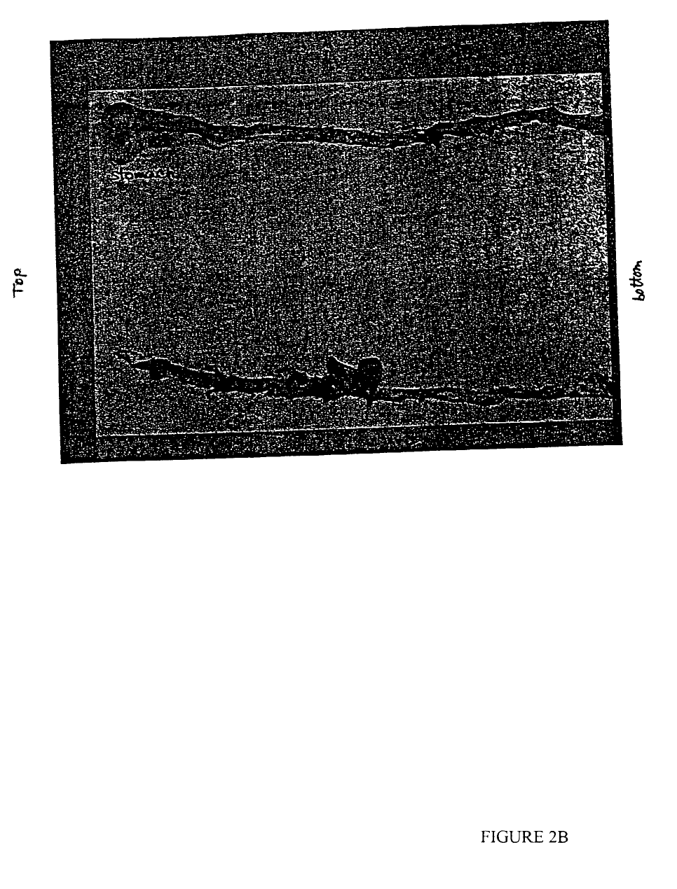

Mice were fasted overnight and then orally administered with either the FS30D coated OVA or L30D coated OVA. Three hours after feeding, mice were sacrificed and the gastrointestinal tract was examined for the presence of intact coated particles. Portions of the intestinal tract of a mouse orally administered with FS30D coated particles is shown in FIG. 2A. Arrows in the figure point to areas where particles were seen. FIG. 2B shows the intestinal tract of a mouse orally administered with L30D coated particles, including the stomach and colon. This figure shows that no L30D coated particles were seen along the entire length of the GI tract 3 hours after feeding. The results suggest that FS30D coated particles dissociate in the lower portion of the gut while L30D coated particles dissociate in the duodenum.

### EXAMPLE 3

Humoral Response of Mice Orally Administered with L30D Coated Particles Versus FS30D Coated Particles.

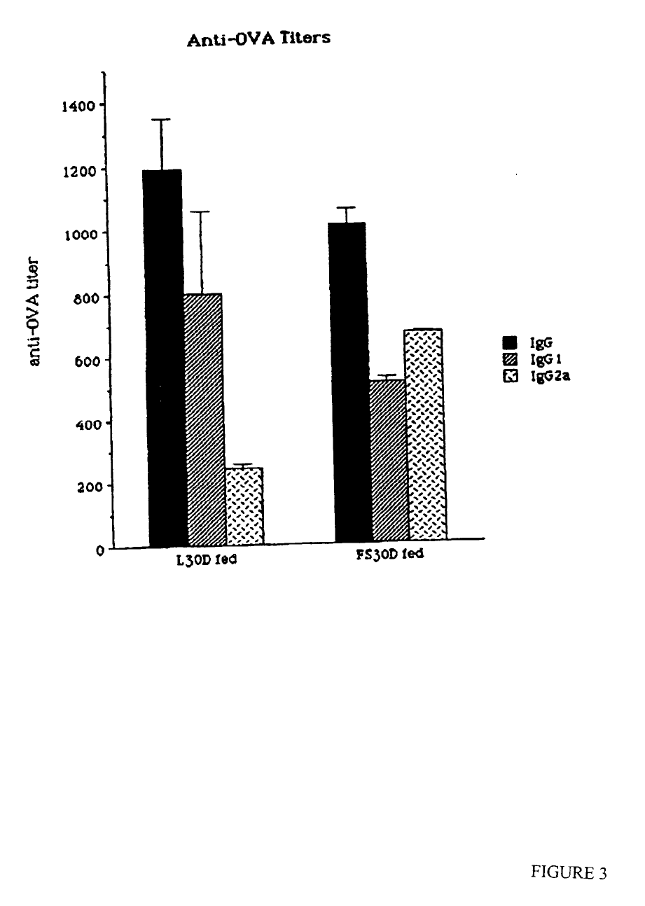

Mice were orally administered with either L30D or FS30D coated particles containing 1% OVA for 3 consecutive days, and bled 21 days after the last feeding. IgG, IgG1, and IgG2a titers in the serum are shown in FIG. 3. The titer of IgE antibodies in the serum was measured by ELISA. The results indicate that FS30D coated particles promote IgG2a production while L30D coated particles promote IgG1 production.

### EXAMPLE 4

Humoral Response of Mice Orally Administered with L30D or FS30D Coated Particles and Challenged with IFA or CFA Subcutaneously.

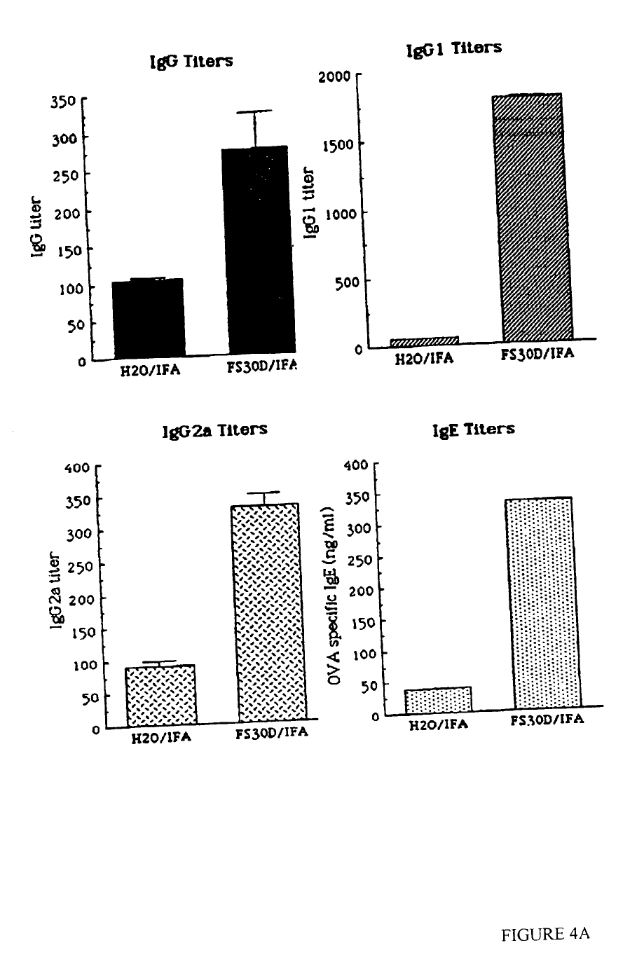

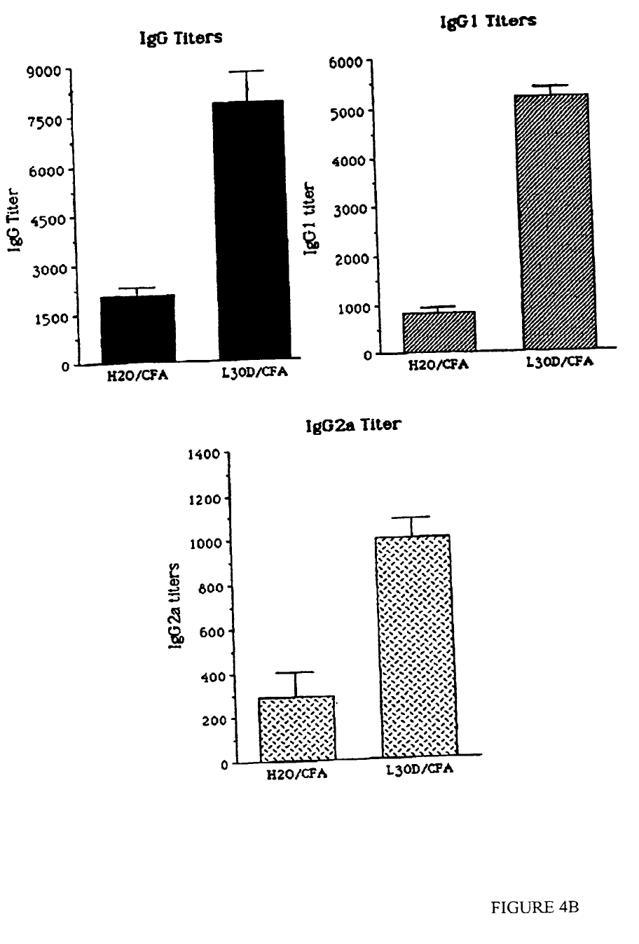

Mice were orally administered L30D coated OVA particles, FS30D coated OVA particles, or just water for three consecutive days. 10 days after the last feeding, the mice were immunized subcutaneously in the footpad with OVA in complete Freund adjuvant (CFA) or OVA in incomplete Freund's adjuvant (IFA). The mice were bled 10 days after the immunization. Titers of IgG, IgG1, IgG2a, and IgE from mice orally administered with FS30D coated particles are shown in FIG. 4A. Titers of IgG, IgG1, and IgG2a from mice orally administered with L30D coated particles are shown in FIG. 4B. The results indicate that priming by the particles is highly effective in inducing the humoral immune response in the mice.

### EXAMPLE 5

T Cell Proliferation from Mice Administered with FS30D- or L30D-Coated Antigen and then Challenged with IFA or CFA Subcutaneously in the Footpad.

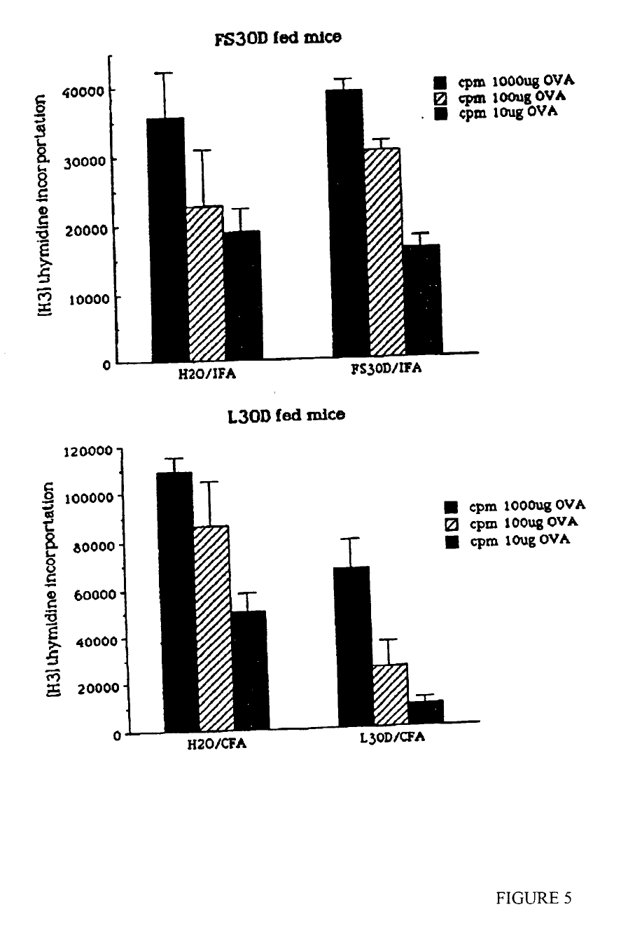

Mice were orally administered with L30D-coated OVA, FS30D-coated OVA, or just water for 3 consecutive days. 10 days after the last feeding, the mice were immunized subcutaneously in the footpad with OVA in complete Freund adjuvant (CFA) or OVA in incomplete Freund's adjuvant (IFA). 10 days after the immunization, the mice were sacrificed and popliteal lymph nodes were collected by standard procedures. Lymphocytes were plated at 6.times.10.sup.6 cells/well in a 96 well flat bottom plate. Cells were cultured 48 hours at 37.degree. C. in RPMI/5% FBS containing 0, 10, 100, or 1000 .mu.g OVA, respectively. Cells were then pulsed for 12 hours with 1 .mu.Ci of [.sup.3H] thymidine. Cells were harvested onto glass fiber filtermats using a Skatron multiple automated sample harvester. FIG. 5 shows the comparison of [.sup.3H] thymidine incorporation in experimental and control mice. The results indicate that there is a decreased T cell proliferation when L30D coated particles are administered, while feeding of FS30D coated particles do not lead to a decrease but possibly an increase in T cell proliferation.

### EXAMPLE 6

Cytokine Analysis of Mice Orally Administered with L30D or FS30D Coated Particles and Challenged with CFA or IFA Subcutaneously

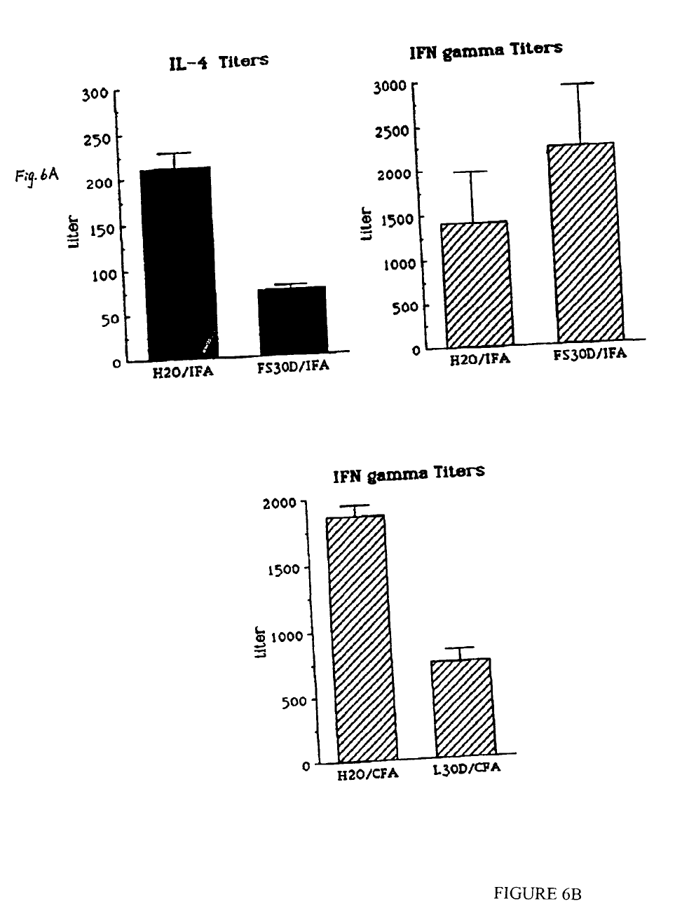

Mice were orally administered with FS30D-coated OVA particles, L30D-coated OVA particles, or just water. 10 days later, the mice were immunized subcutaneously in the footpad with OVA in IFA or OVA in CFA. 10 days after immunization, the mice were sacrificed and spleens were collected accordingly to standard procedures. FIG. 6A shows IF-4 and IFN.gamma. levels in mice orally administered with water or FS30D coated OVA particles. FIG. 6B shows IFN.gamma. levels in mice orally administered with water or L30D-coated OVA particles. The results demonstrate that FS30D-coated particles enhances IFN.gamma. production, a Th1 regulated cytokine, while L30D-coated particles enhances IL-4 production, a Th2 regulated cytokine.

The preceding has been a description of the present invention along with the preferred method currently known of practicing the invention. While there are many minor modifications that can be made without departing from the scope of the present invention, the scope of the present invention should be defined by the appended claims.

All publications, figures, patents and patent applications cited herein are hereby expressly incorporated by reference for all purposes to the same extent as if each was so individually denoted.

1. A method of inducing a type of T helper lymphocyte-regulated immune response in a subject comprising administering to said subject an immunogenic composition comprising at least one immunogen, wherein said immunogen is delivered to a preselected region of the gastrointestinal tract of said subject and induces said type of immune response, wherein said type of immune response is dependent on said region of the gastrointestinal tract.

2. The method of claim 1, wherein said immunogen is selected from the group consisting of an allergen, a killed bacterium or a bacterial component, a killed virus or a viral component, a peptide, a protein fragment, a protein or glycoprotein, a gene, a gene fragment, a DNA, an RNA, a polysaccharide or lipopolysaccharide and combinations thereof.

3. The method of claim 1, wherein said immunogen is selected from the group consisting of a self protein, an altered self protein or peptides or fragments associated with self proteins, wherein said subject has autoimmune disease.

4. The method of claim 1, wherein said immunogen is encapsulated with a coating selected from the group consisting of an aqueous enteric coating, a timed-release coating, and a controlled-release coating, wherein said immunogenic composition is administered orally.

5. The method of claim 1, wherein said immunogen is encapsulated with an acrylic polymeric enteric coating and said region of the gastrointestinal tract is dependent on the composition of said enteric coating.

6. The method of claim 1, wherein said immunogen is encapsulated with both an enteric coating and an aqueous timed-release coating or controlled-release coating, wherein said region of the gastrointestinal tract is dependent on the composition of said timed-release coating or controlled-release coating.

7. The method of claim 5, wherein said immunogenic composition further comprises an adjuvant.

8. The method of claim 5, wherein said immunogenic composition further comprises a mucoadhesive agent for adhering the immunogen to the gastrointestinal tract wall.

9. The method of claim 5, wherein said immunogen is microencapsulated on particles of a pharmaceutically inert material.

10. The method of claim 9, wherein said immunogen is microencapsulated on said inert material along with a stabilizing sugar and a binding agent to bind the immunogen to said inert material.

11. The method of claim 5, wherein said enteric coating dissolves in said region of the gastrointestinal tract, whereby said immunogen is released to said region of the gastrointestinal tract.

12. The method of claim 11, wherein said immunogen is an allergen.

13. The method of claim 11, wherein said enteric coating is selected from the group consisting of a trans-intestinal release coating and a duodenal-release coating, and said immune response is selected from the group consisting of a T helper 1 cell regulated immune response and a T helper 2 cell regulated immune response.

14. The method of claim 13, wherein said enteric coating is Eudragit® FS30D, wherein said region of the gastrointestinal tract is jejunum, ileum, colon, or rectum.

15. The method of claim 14, wherein said immune response is a predominantly T helper 1 cell regulated immune response.

16. The method of claim 13, wherein said enteric coating is Eudragit® L30D, wherein said region of the gastrointestinal tract is duodenum.

17. The method of claim 16, wherein said immune response is a predominantly T helper 2 cell regulated immune response.

18. A method of inducing multiple types of immune responses in a subject comprising administering to said subject a plurality of immunogenic compositions each composition comprising at least one immunogen, wherein the immunogen in each said immunogenic composition is delivered to a different preselected region of the gastrointestinal tract of said subject and induces a different type of immune response, wherein said type of immune response is dependent on said region of the gastrointestinal tract.

19. The method of claim 18, wherein said immunogen is encapsulated with a coating selected from the group consisting of an aqueous enteric coating, a timed-release coating, and a controlled-release coating, wherein said immunogenic compositions are administered orally.

20. The method of claim 18, wherein said immunogen is encapsulated with an acrylic polymeric enteric coating, wherein said immunogenic compositions are administered orally, and said region of the gastrointestinal tract is dependent on the composition of said enteric coating.

21. The method of claim 20, wherein said enteric coating dissolves in said gastrointestinal tract, whereby said immunogen is released to said gastrointestinal tract.

22. The method of claim 21, wherein said enteric coating is selected from the group consisting of a trans-intestinal release coating and a duodenal-release coating, and said immune response is selected from the group consisting of a T helper 1 cell regulated immune response and a T helper 2 cell regulated immune response.

23. The method of claim 22, wherein said trans-intestinal release coating is Eudragit® FS30D and said duodenal-release coating is Eudragit® L30D.

24. A method of orally inducing immune responses in a subject with an immunogen that is acid labile at any pH below 7.0, comprising administering to said subject said immunogen that is encapsulated with an enteric coating, wherein said enteric coating releases said immunogen only at a region of the gastrointestinal tract in which said immunogen is stable.

25. The method of claim 24, wherein said enteric coating is Eudragit® FS30D.

26. The method of claim 24, wherein said region of the gastrointestinal tract is jejunum, ileum, colon, or rectum.

27. The method of claim 24, wherein said immunogen is an influenza viral antigen.
